(19) United States
(12) Patent Application Publication
(10) Pub. No.: US 2004/0266833 A1
(43) Pub. Date: Dec. 30, 2004

(54) Novel use of certain insulin sensitizers or ppar-gamma agonists

(76) Inventor: Robin Edwin Buckingham, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/416,599

(22) PCT Filed: Nov. 14, 2001
(86) PCT No.: PCT/GB01/05044

(30) Foreign Application Priority Data
Nov. 14, 2000 (GB) 0027783.0

(51) Int. Cl. A61K 31/4439; A61K 31/426

(52) U.S. Cl. 514/342; 514/369

(57) ABSTRACT

A use of certain insulin sensitiser or a PPARγ agonist such as a compound of formula (I) or a tautomeric form thereof and/or a pharmaceutically acceptable salt thereof, and/or a pharmaceutically acceptable solvate thereof, wherein: A¹ represents a substituted or unsubstituted aromatic heterocyclyl group; R¹ represents a hydrogen atom, an alkyl group, an acyl group, an aralkyl group, wherein the aryl moiety may be substituted or unsubstituted, or a substituted or unsubstituted aryl group; R² and R³ each represent hydrogen, or R² and R³ together represent a bond; A² represents a benzene ring having in total up to five substituents; and n represents an integer in the range of from 2 to 6, for the manufacture of a medicament for treatment and/or prophylaxis of diseases associated with loss of bone mass, such as osteoporosis and related osteopenic diseases, Paget's disease, hyperparathyroidism and related diseases.

This invention relates to novel use of certain an insulin sensitisers and PPARγ agonists, such as certain substituted thiazolidinedione derivatives and of pharmaceutical compositions containing such compounds.

European Patent Applications, Publication Numbers 0008203, 0139421, 0155845, 0177353, 0193256, 0207581 and 0208420 relate to thiazolidinedione derivatives which are disclosed as having hypoglycaemic and hypolipidaemic activity. Chem. Pharm. Bull 30 (10) 3580-3600 also relates to certain thiazolidinedione derivatives having hypoglycaemic and hypolipidaemic activities.

European Patent Application, Publication Number 0306228 discloses certain substituted thiazolidinedione derivatives of formula (A):

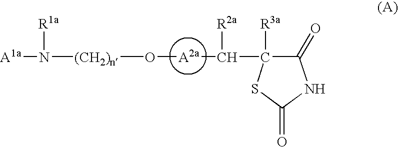

or a tautomeric form thereof and/or a pharmaceutically acceptable salt thereof, and/or a pharmaceutically acceptable solvate thereof, wherein:

A^{1a} represents a substituted or unsubstituted aromatic heterocyclyl group;

R^{1a} represents a hydrogen atom, an allyl group, an acyl group, an aralkyl group, wherein the aryl moiety may be substituted or unsubstituted, or a substituted or unsubstituted aryl group;

R^{2a} and R^{3a} each represent hydrogen, or R^{2a} and R^{3a} together represent a bond;

A^{2a} represents a benzene ring having in total up to five substituents; and

n represents an integer in the range of from 2 to 6. Such compounds are disclosed inter alia as being useful for the treatment and/or prophylaxis of cardiovascular disease and certain eating disorders.

European Patent application, publication number 0783888 discloses the use of troglitazone and certain thiazolidinediones for the treatment of osteoporisis. EP0783888 defines the said certain thiazolidines by use of a formula (I) defined therein. The compounds of formula (I) of EP0783888 are referred to herein as “the compounds of formula (A)”. The disclosures of EP0783888 are incorporated herein by reference.

It has now surprisingly been discovered that the compounds of EP0306228 are indicated to be of particular use of particular use in the treatment and/or prophylaxis of diseases associated with loss of bone mass, such as osteoporosis and related osteopenic diseases, Paget's disease, hyperparathyroidism and related diseases Accordingly, the present invention provides the use of an insulin sensitiser, such as a compound of formula (I):

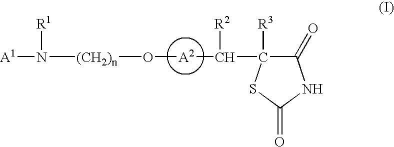

or a tautomeric form thereof and/or a pharmaceutically acceptable salt thereof, and/or a pharmaceutically acceptable solvate thereof, wherein:

A¹ represents a substituted or unsubstituted aromatic heterocyclyl group;

R¹ represents a hydrogen atom, an alkyl group, an acyl group, an aralkyl group, wherein the aryl moiety may be substituted or unsubstituted, or a substituted or unsubstituted aryl group;

R² and R³ each represent hydrogen, or R² and R³ together represent a bond;

A² represents a benzene ring having in total up to five substituents; and

n represents an integer in the range of from 2 to 6, for the manufacture of a medicament for treatment and/or prophylaxis, especially treatment, of diseases associated with loss of bone mass, such as osteoporosis and related osteopenic diseases, Paget's disease, hyperparathyroidism and related diseases

In a further aspect there is provided a method for the treatment and/or prophylaxis, especially the treatment, of diseases associated with loss of bone mass, such as osteoporosis and related osteopenic diseases, Paget's disease, hyperparathyroidism and related diseases, which method comprises the administration of an effective, non-toxic amount of an insulin sensitiser, such as a compound of the above defined formula (I) or a tautomeric form thereof and/or a pharmaceutically acceptable salt thereof, and/or a pharmaceutically acceptable solvate thereof.

There is also provided a pharmaceutical composition for use in the treatment and/or prophylaxis, especially the treatment, of diseases associated with loss of bone mass, such as osteoporosis and related osteopenic diseases, Paget's disease, hyperparathyroidism and related diseases, which composition comprises an insulin sensitiser, such as a compound of the above defined formula (I) or a tautomeric form thereof and/or a pharmaceutically acceptable salt thereof, and/or a pharmaceutically acceptable solvate thereof, and a pharmaceutically acceptable carrier therefor.

A particular disease associated with loss of bone mass is osteoporosis. A particular disease associated with loss of bone mass is Paget's disease. A particular disease associated with loss of bone mass is hyperparathyroidism.

A suitable insulin sensitiser is a compound of the above defined formula (I).

Suitable aromatic heterocyclyl groups include substituted or unsubstituted, single or fused ring aromatic heterocyclyl groups comprising up to 4 hetero atoms in each ring selected from oxygen, sulphur or nitrogen.

Favoured aromatic heterocyclyl groups include substituted or unsubstituted single ring aromatic heterocyclyl groups having 4 to 7 ring atoms, preferably 5 or 6 ring atoms.

In particular, the aromatic heterocyclyl group comprises 1, 2 or 3 heteroatoms, especially 1 or 2, selected from oxygen, sulphur or nitrogen.

Suitable values for A¹ when it represents a 5-membered aromatic heterocyclyl group include thiazolyl and oxazolyl, especially oxazolyl.

Suitable values for A¹ when it represents a 6-membered aromatic heterocyclyl group include pyridyl or pyrimidinyl.

Suitably R² and R³ each represent hydrogen.

Preferably, A¹ represents a moiety of formula (a), (b) or (c):

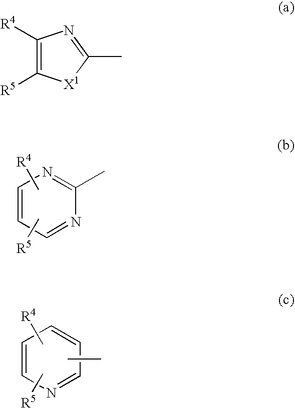

wherein: R⁴ and R⁵ each independently represents a hydrogen atom, an alkyl group or a substituted or unsubstituted aryl group or when R⁴ and R⁵ are each attached to adjacent carbon atoms, then R⁴ and R⁵ together with the carbon atoms to which they are attached form a benzene ring wherein each carbon atom represented by R⁴ and R⁵ together may be substituted or unsubstituted; and in the moiety of formula (a) X represents oxygen or sulphur.

Aptly, A¹ represents a moiety of the abovedefined formula (a).

Aptly, A¹ represents a moiety of the abovedefined formula (b).

Aptly, Al represents a moiety of the abovedefined formula (c).

In one favoured aspect R⁴ and R⁵ together represent a moiety of formula (d):

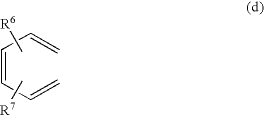

wherein R⁶ and R⁷ each independently represent hydrogen, halogen, substituted or unsubstituted alkyl or alkoxy.

Suitably, R⁶ and R⁷ each independently represent hydrogen, halogen, alkyl or alkoxy.

Favourably, R⁶ represents hydrogen. Favourably, R⁷ represents hydrogen.

Preferably, R⁶ and R⁷ both represent hydrogen.

In a further favoured aspect R⁴ and R⁵ each independently represent hydrogen, alkyl or a substituted or unsubstituted phenyl group and more favourably, R⁴ and R⁵ each independently represent hydrogen, alkyl or phenyl.

Preferably, for the moiety of formula (a), R⁴ and R⁵ together represent the moiety of formula (d).

Preferably, for the moieties of formula (b) or (c), R⁴ and R⁵ both represent hydrogen.

It will be appreciated that the five substituents of A² include three optional substituents. Suitable optional substituents for the moiety A² include halogen, substituted or unsubstituted alkyl or alkoxy.

Favourably, A² represents a moiety of formula (e):

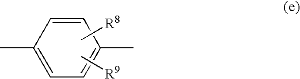

wherein R⁸ and R⁹ each independently represent hydrogen, halogen, substituted or unsubstituted alkyl or alkoxy.

Suitably, R⁸ and R⁹ each independently represent hydrogen, halogen, alkyl or alkoxy. Preferably, R⁸ and R⁹ each represent hydrogen.

Favourably, X represents oxygen. Favourably, X represents sulphur.

In one preferred aspect the present invention provides a class of compounds, which fall wholly within the scope of formula (I), of formula (II):

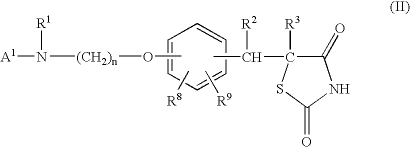

or a tautomeric form thereof, and/or a pharmaceutically acceptable salt thereof and/or a pharmaceutically acceptable solvate thereof, wherein A¹, R¹, R², R³, and n are as defined in relation to formula (I) and R⁸ and R⁹ are as defined in relation to formula (e).

Suitably, n represents an integer 2, 3 or 4, notably 2 or 3 and especially 2.

Suitably, R¹ represents hydrogen, alkyl, acyl, especially acetyl, or benzyl.

When R¹ represents an alkyl group, examples of such alkyl groups include methyl and isopropyl. Preferably, R¹ represents a methyl group.

As indicated above a compound of formula (I) may exist in one of several tautomeric forms, all of which are encompassed by the present invention. It will be appreciated that the present invention encompasses all of the isomeric forms of the compounds of formula (I) and the pharmaceutically acceptable salts thereof, including any stereoisomeric forms thereof, whether as individual isomers or as mixtures of isomers.

Suitable substituents for any heterocyclyl group include up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

When used herein the term ‘aryl’ includes phenyl and naphthyl optionally substituted with up to five, preferably up to three, groups selected from halogen, alkyl, phenyl, alkoxy, haloalkyl, hydroxy, amino, nitro, carboxy, alkoxycarbonyl, alkoxycarbonylalkyl, alkylcarbonyloxy, or alkylcarbonyl groups.

When used herein the term ‘halogen’ refers to fluorine, chlorine, bromine and iodine; preferably chlorine. When used herein the terms ‘alkyl’ and ‘alkoxy’ relate to groups having straight or branched carbon chains, containing up to 12 carbon atoms.

When used herein the term ‘acyl’ includes alkylcarbonyl groups.

Suitable alkyl groups are C₁₋₁₂ alkyl groups, especially C₁₋₆ alkyl groups e.g. methyl, ethyl, n-propyl, iso-propyl, n-butyl, isobutyl or tert-butyl groups.

Suitable substituents for any alkyl group include those indicated above in relation to the term “aryl”.

Suitable pharmaceutically acceptable salts include salts of the thiazolidinedione moiety, and, where appropriate, salts of carboxy groups.

Suitable pharmaceutically acceptable salts of the thiazolidinedione moiety include metal salts especially alkali metal salts such as the lithium, sodium and potassium salts.

Suitable pharmaceutically acceptable salts of carboxy groups include metal salts, such as for example aluminium, alkali metal salts such as sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroabietylamine, N,N-bisdehydroabietylamine, glucamine, N-methylglucamine or bases of the pyridine type such as pyridine, collidine or quinoline.

Suitable pharmaceutically acceptable solvates include hydrates.

The salts and/or solvates of the compounds of formula (I) may be prepared and isolated according to conventional procedures for example sodium salts may be prepared by using sodium methoxide in methanol.

Suitable pharmaceutically acceptable salts of the thiazolidinedione moiety include metal salts especially alkali metal salts such as the lithium, sodium and potassium salts.

A preferred compound of formula (I) is 5-[4-[2-(N-methyl-N-(2-pyridyl)amino)ethoxy]benzyl]thiazolidine-2,4-dione (herein after also refered to as “Compound (I)”) or a tautomeric form thereof and/or a pharmaceutically acceptable salt thereof and/or a pharmaceutically acceptable solvate thereof.

A preferred salt of Compound (I) is a maleate salt as disclosed in International Application, publication number WO94/05659.

As is known in the art Compound (I) is a PPARγ agonist. Thus the invention also includes the use of a PPARγ agonist, in the manufacture of a medicament for the treatment and/or prophylaxis, especially the treatment, of diseases associated with loss of bone mass, such as osteoporosis and related osteopenic diseases, Paget's disease, hyperparathyroidism and related diseases.

In a further aspect there is provided a method for the treatment and/or prophylaxis, especially the treatment, of diseases associated with loss of bone mass, such as osteoporosis and related osteopenic diseases, Paget's disease, hyperparathyroidism and related diseases, which method comprises the administration of an effective, non-toxic amount of a PPARγ agonist.

There is also provided a pharmaceutical composition for use in the treatment and/or prophylaxis, especially the treatment, of diseases associated with loss of bone mass, such as osteoporosis and related osteopenic diseases, Pagefs disease, hyperparathyroidism and related diseases, which composition comprises a PPARγ agonist and a pharmaceutically acceptable carrier therefor.

The above mentioned insulin sensitisers do not include troglitazone or the compounds of formula (A) or pharmaceutically acceptable derivatives thereof.

The above mentioned PPARγ agonists do not include troglitazone or the compounds of formula (A) or pharmaceutically acceptable derivatives thereof. Suitable insulin sensitisers or PPARγ agonists are thiazolidinediones.

Suitable insulin sensitisers or PPARγ agonists are inslin sensitisers or PPARγ agonists other than thiazolidinediones.

Suitable non-thiazolidinedione insulin sensitisers include the compounds of formula (I) of International application, publication number WO 97/31907 or a pharmaceutically acceptable derivative thereof. A particular compound of WO 97/31907 (or EP0888317) is 2(S)-(2-benzoyl-phenylamino)-3-{4-[2-5-methyl-2-phenyl-oxazol-4-yl)-ethoxy]-phenyl}-propionic acid or a pharmaceutically acceptable derivative thereof, such as a pharmaceutically acceptable salt or pharmaceutically acceptable solvate thereof.

The contents of WO 97/31907 (or EP0888317) are included herein by reference.

The insulin sensitisers or PPARy agonists mentioned herein are prepared according to methods known in the art including those dislcoed in the above mentioned publications. Thus a compound of above defined formula (I) such as Compound (I), or the tautomeric form thereof, and/or a pharmaceutically acceptable salt thereof, and/or a pharmaceutically acceptable solvate thereof, may be prepared using the processes described in EP 0306228. The contents of EP 0306228 are incorporated herein by reference

As mentioned above the compounds of the invention are indicated as having useful therapeutic properties:

The insulin sensitisers or PPARy agonists of the invention such as a compound of formula (I), or a tautomeric form thereof and/or a pharmaceutically acceptable salt thereof and/or a pharmaceutically acceptable solvate thereof, may be administered per se or, preferably, as a pharmaceutical composition also comprising a pharmaceutically acceptable carrier.

As used herein the term ‘pharmaceutically acceptable’ embraces compounds, compositions and ingredients for both human and veterinary use: for example the term ‘pharmaceutically acceptable salt’ embraces a veterinarily acceptable salt.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

The present invention further provides a method for the treatment of diseases associated with loss of bone mass, such as osteoporosis and related osteopenic diseases, Paget's disease, hyperparathyroidism and related diseases, in a human or non-human mammal, which comprises administering an effective, non-toxic, amount of a compound of formula (I), or a tautomeric form thereof and/or a pharmaceutically acceptable salt thereof and/or a pharmaceutically acceptable solvate thereof, to a human or non-human mammal in need thereof.

Conveniently, the active ingredient may be administered as a pharmaceutical composition hereinbefore defined, and this forms a particular aspect of the present invention.

Most suitably the composition will be formulated in unit dose form. Such unit dose will normally contain an amount of the active ingredient in the range disclosed in the above mentioend publications, for example for a compound of the above defined formula (I) such as Compound (I), in the range of from 0.1 to 1000 mg, more usually 0.1 to 500 mg, and more especially 0.1 to 250 mg. The unit dosages of each of the compounds specifically mentioned herein In the above mentioned treatments the insulin sensitisers or PPARγ agonists of the invention such as the compound of the general formula (I), or a tautomeric form thereof and/or a pharmaceutically acceptable salt thereof and/or a pharmaceutically acceptable solvate thereof, may be taken in doses, such as those described in the above mentioned publications including, one to six times a day in a manner such that the total daily dose for a 70 kg adult will generally be in the range of from 0.1 to 6000 mg, and more usually about 1 to 1500 mg.

Particularly, the method comprises the administration of 2 to 4, 4 to 8 or 8 to 12 mg of Compound (I) per day.

Particularly, the method comprises the administration of 2 to 4 mg of Compound (I), especially when administered per day.

Particularly, the method comprises the administration of 4 to 8 mg of Compound (I), especially when administered per day.

Particularly, the method comprises the administration of 8 to 12 mg of Compound (I), especially when administered per day.

Preferably, the method comprises the administration of 2 mg of Compound (I), especially when administered per day.

Preferably, the method comprises the administration of 4 mg of Compound (I), especially when administered per day.

Preferably, the method comprises the administration of 8 mg of Compound (I), especially when administered per day.

A further suitable compound for use in the present treatment is the thiazolidinedione insulin sensitiser 5-[4-[2-(5-ethylpyridin-2-yl)ethoxy]benzyl] thiazolidine-2,4-dione (or pioglitazone). Methods of preparation and formulation of this compound are known in the art, as for example is disclosed in European Application, Publication Number EP 0749751.

Suitable unit dosages of the actives include all the known doses for these compounds as described or referred to in reference texts such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

The compositions are also formulated according to conventional methods, such as those disclosed in standard reference texts including the above mentioned reference texts and Harry's Cosmeticology (Leonard Hill Books).

The activity of compounds as agents effective in the treatment or prophylaxis of diseases associated with loss of bone mass are assessed using known methodology for example those disclosed in Wronski, T. J., Lowry, P. L., Walsh, C. C. and Ignaszewski L. A. 1985 “Skeletal alterations in ovariectomized rats.” Calcified Tissue International 37:324-328). or Dunstan, C. R. and Boyce B. F. Animal models for the investigation of the action of factors on bone metabolism In: Methods in Bone Biology, eds: T. R. Arnett and B. Henderson, Chapman and Hall, 1998, pp 290-303.

1-7. (canceled)

8. A method for the treatment or prophylaxis of a disease associated with loss of bone mass in a human or non-human mammal comprising administering an effective, non-toxic amount of a compound, wherein said compound is an insulin sensitizer or a PPARy agonist, to a human or non-human mannal in need thereof, wherein said compound is not trogliazone.

9. A method according to claim 8, wherein the disease associated with loss of bone mass is osteoporosis.

10. A method according to claim 8, wherein the disease associated with loss of bone mass is Paget's disease.

11. A use according to claim 8, wherein the disease associated with loss of bone mass is hyperparathyroidism.

12. A method according to claim 8, wherein said compound is a compound according to formula (I) 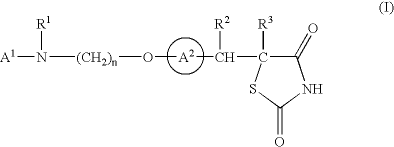
or a tautomeric form thereof, or a pharmaceutically acceptable salt thereof, or a pharmaceutically acceptable solvate thereof, wherein:
A¹ represents a substituted or unsubstituted aromatic heterocyclyl group;
R¹ represents a hydrogen atom, an alkyl group, an acyl group, an aralkyl group, wherein the aryl moiety may be substituted or unsubstituted, or a substituted or unsubstituted aryl group;
R² and R³ each represent hydrogen, or R² and R³ together represent a bond;
A² represents a benzene ring having in total up to five substituents; and
n represents an integer in the range of from 2 to 6;
wherein said compound of formula (I) is not troglitazone, or a tautomeric form thereof, or a pharmaceutically acceptable salt thereof, or a pharmaceutically acceptable solvate thereof.

13. A method according to claim 12, wherein said compound according to formula (I) is 5-[4-[2-(N-methyl-N-(2-pyridyl)amino)ethoxy]benzyl]thiazolidine-2,4-dione, or a tautomeric form thereof, or a pharmaceutically acceptable salt thereof, or a pharmaceutically acceptable solvate thereof.

14. A method according to claim 13, wherein said 5-[4-[2-(N-methyl-N-(2-pyridyl)amino)ethoxy]benzyl]thiazolidine-2,4-dione is in the form of a maleate salt.

15. A method according to claim 8, wherein said compound is selected from the group: 2(S)-(2-benzoyl-phenylamino)-3-{4-[2-5-methyl-2-phenyl-oxazol-4-yl)-ethoxy]-phenyl}-propionic acid, 5-[4-[2-(5-ethylpyridin-2-yl)ethoxy]benzyl] thiazolidine-2,4-dione, a pharmaceutically acceptable salt thereof and a pharmaceutically acceptable solvate thereof.

16. A method according to claim 13, which comprises administering 2 to 4 mg, 4 to 8 mg, or 8 to 12 mg of said 5-[4-[2-(N-methyl-N-(2-pyridyl)amino)ethoxy]benzyl]thiazolidine-2,4-dione, or a tautomer thereof.

17. A method according to claim 16, which comprises administering 2 to 4 mg of said 5-[4-[2-(N-methyl-N-(2-pyridyl)amino)ethoxy]benzyl]thiazolidine-2,4-dione, or a tautomer thereof.

18. A method according to claim 16, which comprises administering 4 to 8 mg of said 5-[4-[2-(N-methyl-N-(2-pyridyl)amino)ethoxy]benzyl]thiazolidine-2,4-dione, or a tautomer thereof.

19. A method according to claim 16, which comprises administering 8 to 12 mg of said 5-[4-[2-(N-methyl-N-(2-pyridyl)amino)ethoxy]benzyl]thiazolidine-2,4-dione, or a tautomer thereof.

20. A method according to claim 16, which comprises administering 2 mg of said 5-[4-[2-(N-methyl-N-(2-pyridyl)amino)ethoxy]benzyl]thiazolidine-2,4-dione, or a tautomer thereof.

21. A method according to claim 16, which comprises administering 4 mg of said 5-[4-[2-(N-methyl-N-(2-pyridyl)amino)ethoxy]benzyl]thiazolidine-2,4-dione, or a tautomer thereof.
